(19) United States
(12) Patent Application Publication
(10) Pub. No.: US 2022/0028564 A1
(43) Pub. Date: Jan. 27, 2022

(54) DEVICE FOR MEASURING ENVIRONMENTAL FACTORS AND RECOMMENDING PERSONALIZED MITIGATING REGIMEN

(71) Applicant: L'Oreal, Paris (FR)

(72) Inventors: David B. Kosecoff, San Francisco, CA (US); Rafal Pielak, 604 Mission Street, CA (US); Haruna Peyret, San Francisco, CA (US); Guive Balooch, Clark, NJ (US)

(73) Assignee: L'Oreal, Paris (FR)

(21) Appl. No.: 16/938,816

(22) Filed: Jul. 24, 2020

(51) Int. Cl. G16H 70/20; G16H 40/63

(52) U.S. Cl. CPC G16H 70/20; G01N 33/0004; G16H 40/63

(57) ABSTRACT

A computer system and the computer-implemented method of generating and providing skin or hair care product recommendations to a subject, wherein the method comprises determining, by a computing device, more than one pollutant exposure amount for the subject; determining, by the computing device, a target exposure limit for each pollutant; determining, by the computing device, pollutants to which the subject has the highest exposure; and providing, by the computing device, at least one skin or hair care product recommendation to the subject, wherein the recommendation is based on the pollutants to which the subject has the highest exposure.

### BACKGROUND

### SUMMARY

Personal sensor product that measures harmful light and air pollution exposure and recommends more personalized set of personal skin and hair products/personal regimen to mitigate environmental effects, help with employee and athlete wellness, give user a Lifetime Exposure level, when it's safe to go outside or remove protective equipment.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 a schematic diagram that illustrates a non-limiting example embodiment of a system for generating and providing product recommendations to a subject according to various aspects of the present disclosure;

FIG. 2 is a block diagram that illustrates a non-limiting example embodiment of a system that includes a mobile computing device and a server computing device according to various aspects of the present disclosure;

FIG. 3 is a block diagram that illustrates a non-limiting example embodiment of a computing device appropriate for use as a computing device with embodiments of the present disclosure; and

FIG. 4 is a flowchart that illustrates a non-limiting example embodiment of a method of generating and providing recommendations to a subject according to various aspects of the present disclosure.

### DETAILED DESCRIPTION

High levels of air pollutants indoors or outdoors can contribute to health issues that detrimentally affect one's performance in one's occupation or athletic activity. A wellness program can include proactive regimens and recommendations to lessen the risk of being adversely affected by pollutants. As used herein, “pollutant” denotes any elements, molecules, particles, radiation, electromagnetic energy, environmental factors and the like that are tracked according to this disclosure. In some embodiments, a pollutant can have an adverse effect on a person.

In some embodiments, the present disclosure is directed to a computer system and computer-implemented method or App to educate and alert subjects about their exposure (real-time/hourly/daily/lifetime, etc.) to the full range of indoor and outdoor environmental factors that can damage their skin and hair. This is particularly important for people who work in environments that have a high concentration of one or more pollutants or potentially harmful elements.

In some embodiments, the present disclosure is directed to a computer system and computer-implemented method to provide subjects with more information on their exposure levels to harmful pollutants as they move through different environments in their daily lives. In some embodiments, the subjects are informed about the harmful effects to allow the subject to purchase a wider range (a set) of skin and hair products that correct or protect against the various effects. In some embodiments, the present disclosure is directed to a computer system and computer-implemented method to guide the subject on skin and haircare products that are tailored to their lifestyle.

Various dedicated sensors already exist for measuring different types of light radiation (i.e. UV-A, UV-B, Blue HEV, IR) and air-borne pollutants (i.e., CO, CO2, NO2, NOx, SO2, O3, PM2.5, PM10, VOC, heavy metals, radiation) as well as environmental factors, such as temperature and humidity, by personal, wearable, and mobile devices. The amount of light radiation and air-borne pollutants at given locations is also available for downloading from various sources on the Internet.

In some embodiments, the amount of pollutant in any environment is transmitted via a wireless technology to a smart device (e.g., a mobile phone) running a corresponding pollutant App. The pollutant App receives these constantly changing values based on the subject's location and integrates them over time to calculate exposure duration, determines whether or not the subject is at risk of harm, and alerts the subject accordingly.

In an embodiment, those pollutants that are trending higher constitute a profile of the subject that the pollutant App can use for making personalized product recommendations, adjusting work schedules to minimize exposure, and structuring exercise routines.

In an embodiment, the pollutant App uses recorded logs in conjunction with internet-based weather forecasts to advise the subject on when it is safe to go outside and for how long. Data collected by the pollutant App, automatically and/or provided by the subject, can represent the subject's ‘lifestyle’ and habits, which is valuable for optimizing help information and product recommendations.

This disclosure relates to a computer system and a computer-implemented method that intelligently calculates and advises, for example, on the levels of harmful pollutants, when it may be more desirable to go outside, when to go inside to minimize exposure or reached the maximum exposure limit, and when to apply a skin or haircare product based on, for example, the type of pollutant or the pollutants to which the subject has the highest exposure. In addition, the computer system and a computer-implemented method additionally can alert a subject when any one or more pollutant has reached a maximum target exposure based on an hourly/daily/weekly/yearly/lifetime exposure.

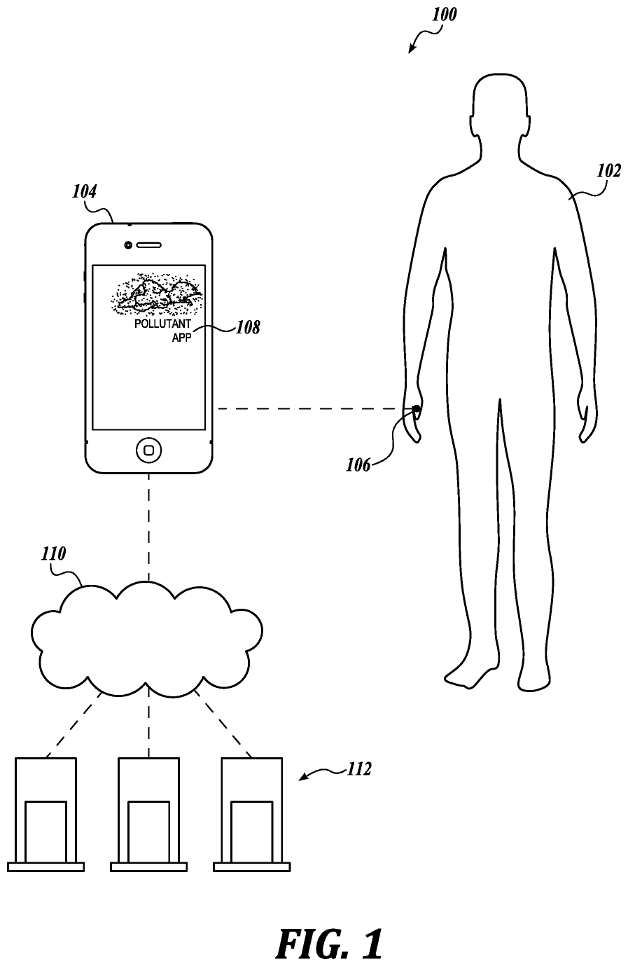

FIG. 1 is a schematic diagram that illustrates a non-limiting example embodiment of a system 100 for tracking a subject's exposure time to one or more pollutants, recommending a personalized set of skin or hair care products depending on the pollutants which the subject is most exposed to, and warning the subject when the exposure to one or more pollutants is reaching the maximum permissible exposure limit, and recommending a skin or haircare product and regimen to prevent or alleviate damage caused by the one or more pollutants.

In the system 100, the subject 102 interacts with a mobile computing device 104. In some embodiments, the mobile computing device 104 may be used to receive exposure data of one or more pollutants from a wearable UV sensor 106 on the subject 102, additionally or alternatively, the data may come from one or more sources on the Internet, for example, online sources can report the air quality for a particular location, for example, online sites can provide the amount of ozone (O3), particulate matter (PM), sulfur dioxide (SO2), nitrogen dioxide (NO2) and nitrogen oxides (NOx).

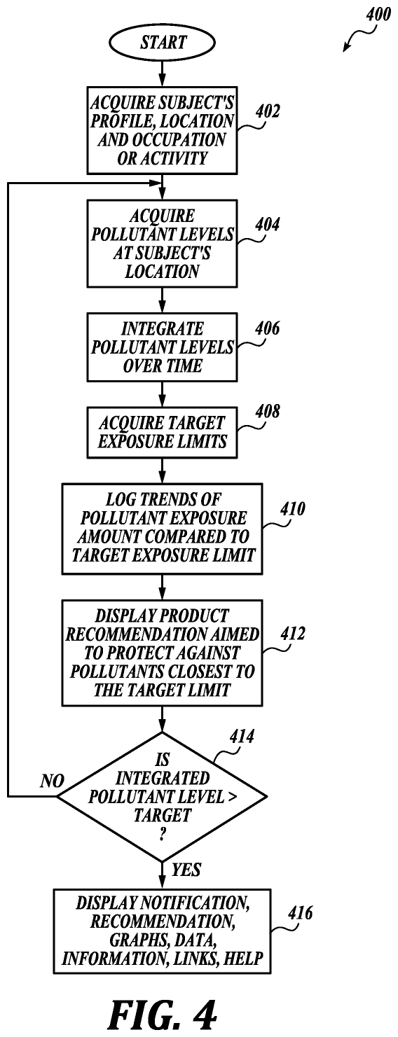

In some embodiments, the mobile computing device 104 is capable of performing the computer-implemented method or App. The subject may start the computer-implemented method by touching the icon 108 on a touch-sensitive display of the mobile computing device 104. The computer-implemented method is further described in connection with FIG. 4.

In some embodiments, the mobile computing device 104 is connected to a remote server computer system 112 comprised of one or more server computers via a network, such as the Internet 110. The network may include any suitable networking technology, including but not limited to a wireless communication technology (including but not limited to Wi-Fi, WiMAX, Bluetooth, 2G, 3G, 4G, 5G, and LTE), a wired communication technology (including but not limited to Ethernet, USB, and FireWire), or combinations thereof.

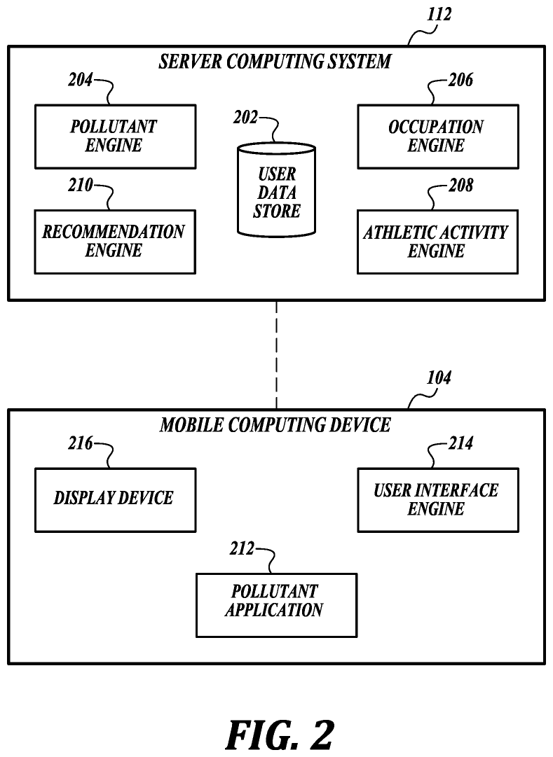

FIG. 2 is a block diagram that illustrates a non-limiting example embodiment of a system that includes the mobile computing device 104 and a server computing system 112 according to various aspects of the present disclosure. In some embodiments, the mobile computing device 104 may be a smartphone. In some embodiments, the mobile computing device 104 may be any other type of computing device having the illustrated components, including but not limited to a tablet computing device or a laptop computing device. In some embodiments, the mobile computing device 104 may not be mobile, but may instead be a stationary computing device, such as a desktop computing device. In some embodiments, the illustrated components of the mobile computing device 104 may be within a single housing. In some embodiments, the illustrated components of the mobile computing device 104 may be in separate housings that are communicatively coupled through wired or wireless connections. The mobile computing device 104 also includes other components that are not illustrated, including but not limited to one or more processors, a non-transitory computer-readable medium, a power source, and one or more communication interfaces.

As shown, the mobile computing device 104 includes, at least, a display device 216, a pollutant Application 212 (pollutant App 212), and a user interface engine 214.

In some embodiments, the display device 216 is an LED display, an OLED display, or another type of display for presenting a user interface. In some embodiments, the display device 216 may be combined with or include a touch-sensitive layer, such that a subject 102 may interact with a user interface presented on the display device 216 by touching the display. In some embodiments, a separate user interface device, including but not limited to a mouse, a keyboard, or a stylus, may be used to interact with a user interface presented on the display device 216.

In some embodiments, the user interface engine 214 is configured to present a user interface on the display device 216 when opening the pollutant App 212. The pollutant App 212 will cause the user interface engine 214 to display a plurality of user interfaces on the display device 216 relating to a computer-implemented method used for the gathering and display of information, including recommending a personalized set of skin or hair care products depending on the pollutants to which the subject has the highest exposure.

In an embodiment, the user interface engine 214 can present the subject with a questionnaire that is useful to elicit information for determining the subject's profile, such as, but not limited to, occupation, daily, weekly, and monthly schedules, athletic activities, the skin and haircare products currently used, hair styling methods currently used, but also provide other options and information.

In some embodiments, the server computing system 112 includes one or more computing devices that each include one or more processors, non-transitory computer-readable media, and network communication interfaces that are collectively configured to provide the illustrated components. In some embodiments, the one or more computing devices that make up the server computing system 112 may be rack-mount computing devices, desktop computing devices, or computing devices of a cloud computing service.

In some embodiments, the server computing system 112 is configured to perform data analytics for determining the pollutant amount as a subject's location changes, integrating the pollutant exposure over time, comparing the pollutant exposure to target exposure levels depending on, for example, occupation and athletic activity, and determining the pollutants to which the subject has the highest exposure, and making product recommendations.

In some embodiments, the mobile computing device 104 is configured to connect to the server computing system 112 in a cloud computing environment. As shown, the server computing system 112 includes a user data store 202, a pollutant engine 204, an occupation engine 206, an athletic activity engine 208, and a recommendation engine 210. In some embodiments, some or all of the components of the user data store 202, pollutant engine 204, occupation engine 206, athletic activity engine 208, and a recommendation engine 210 can reside in the mobile computing device 104.

In some embodiments, the user data store 202 is configured to store records for each subject 102 that uses the system. The records may the subject's profile including medical or personal records, such as age, weight, skin type, hair type, residence, occupation, athletic activities, schedules, product recommendations, descriptions of lifestyle, and/or other information collected or determined by the system.

In some embodiments, the user data store 202 may also contain a database of skin and hair care products, wherein each skin and hair care product is identified by or classified according to one or more attributes. For example, a skin or hair care product can be classified as having one or more of the following attributes: a UV blocker, a moisturizer, a humectant, antioxidant source, hyaluronic acid source, collagen source. In this manner, the recommendation engine 210 can recommend products that more precisely directed to the type of damage caused by a particular pollutant.

In some embodiments, the pollutant engine 204 may be configured to process the data acquired by a wearable pollutant sensor 106 to determine pollutant levels and exposure times of the subject. In some embodiments, the pollutant engine 204 may be configured to process the data acquired by online sources reporting the amount of air pollutants at the given location of the subject. In some embodiments, the pollutant engine 204 may be configured to both process the data acquired by the pollutant sensor 106 and data acquired through online sources. In some embodiments, the pollutant engine 204 may be configured to calculate the amount of pollutant exposure on a minute, hourly, daily, weekly, monthly, yearly, or lifetime basis. In some embodiments, the pollutant engine 204 calculates the pollutant levels by keeping track of a subject's location by global positioning system (GPS) coordinates.

In some embodiments, the pollutant engine 206 is configured to calculate the subject's exposure to one or more pollutants over time, and compare the amount of exposure to the target exposure limits on a minute, hourly, daily, weekly, monthly, yearly, or lifetime basis. In some embodiment, the pollutant engine 206 tracks the highest trending pollutants to which the subject has the highest exposure and adjusts the skin and hair care product recommendations based on the highest trending pollutants.

In an embodiment, not all pollutants can be tracked based on the same scale or units. Therefore, in one embodiment, a pollutant is tracked based on a relative scale compared to all other pollutants. For example, each pollutant can be assigned a target exposure limit. The pollutant engine 204 keeps track of the amount of pollutant exposure as a percentage compared to the target exposure limit of each pollutant. Then, in one embodiment, keeping track of the highest trending pollutants to which the subject has the highest exposure is based on comparing percentages, for example, the percentage of exposure of each pollutant to the target limit of the pollutant. Identifying the highest trending pollutants is done by comparing the percentages of exposure of the pollutants relative to each pollutant's unique target exposure limit.

In some embodiments, target exposure levels can be determined through governmental agencies, such as, for the United States, the Occupational Health and Safety Administration (OSHA). These exposure limits are called permissible exposure limits and are a well-known quantity. In some embodiments, the target exposure limit does not coincide with the PEL as determined by a government agency. In some embodiments, the target exposure limit is the amount of exposure that can result in damage to skin or hair. For example, low humidity and temperature can lead to dry skin. Air-borne pollutants, such as particulate matter (e.g., soot and heavy metals) and volatile organic gases (VOCs) can also lead to dryness and irritation of the scalp and cause weakened hair that can break. Some hair sprays and other products, as well as the styling method (heat) may even exacerbate these conditions. Therefore, the pollutant App 212 can keep track of each subject's profile and pollutant exposure levels and can provide recommendations on product selection, styling methods, skin and haircare regiments that are based on the levels of pollutants that can damage skin and hair, or that can exacerbate damage to skin and hair.

In some embodiments, the pollutant App 212 can use different target exposure limits for the same pollutant. First, there are target exposure limits of pollutants that are determined based on the amount of exposure that can lead to any adverse effect to skin or hair. Second, there are target exposure levels of pollutants that coincide with the PELs of government entities. In the former case, the recommendation engine 210 can provide a personalized set of skin and hair product recommendations, and in the latter case, the recommendation engine 210 can provide warning notices that a pollutant exposure level may exceed a permissible exposure limit, and issue other recommendations, such as leaving the area, donning personal protective equipment, changing or reducing work schedules, changing or reducing athletic practice schedules, and provide other useful help and information.

In some embodiments, the pollutant App 212 does not use the same target exposure limits for each subject. In some embodiments, the pollutant App 212 can adjust the target exposure limit based on each subject's profile. For example, factors in a subject's profile may be used to increase or decrease target exposure limit for a pollutant.

In some embodiments, the occupation engine 206 is used for managing a subject's occupational wellness program. In some embodiments, an occupation wellness program may include information for encouraging healthy behaviors for adopting into company culture. For example, an occupational wellness program may provide help on nutrition, physical fitness, mental health, social activities, stress relief, team building, and set goals to encourage the aforementioned. In some embodiments, an occupational wellness program may further include incentives for encouraging healthy behaviors in company culture. In some embodiments, the occupation engine 206 can calculate the pollutant target exposure limits used by the pollutant engine 206 based on the subject's occupation or based on criteria particular to the subject's profile determined by the subject's unique wellness program. In this manner, product recommendations can also be personalized to the subject. The pollutant target exposure limits can include PELs and other pollutant target exposure limits to prevent or mitigate skin or hair damage caused by certain pollutants.

In some embodiments, the athletic activity engine 208 is used for managing a subject's athlete wellness program. In some embodiments, an athlete wellness program may include incentives for encouraging healthy behaviors for individuals and teams. For example, an athlete wellness program may provide help on nutrition, training and physical fitness, injury recovery, healthcare provider services, and set goals to encourage the aforementioned. In some embodiments, the athletic activity engine 208 can calculate the pollutant target exposure limits used by the pollutant engine 206 based on the subject's athletic activities or sport or based on criteria particular to the subject's profile determined by the subject's unique wellness program. In this manner, product recommendations can be personalized to the subject. The pollutant target exposure limits can include PELs and other pollutant target exposure limits to prevent or mitigate skin or hair damage caused by certain pollutants.

In some embodiments, the recommendation engine 210 is configured to generate recommendations of, at least, skin care products or hair care products for protection against one or more pollutants or for care of damaged skin or hair caused by pollutants. In some embodiments, the recommendation engine 210 provides a set of skin and hair care product recommendations based on the pollutants to which the subject has the highest exposure.

In some embodiments, the recommendation engine 210 can further calculate recommendations based on the subject's profile, such as currently used products and styling methods, and also considers an occupation or athletic wellness program. In this manner, the recommendation engine 210 is able to provide a personalized set of skin and haircare products unique to the subject.

In some embodiments, skin care products can be provided as creams, spray liquids, patches, and the like, and hair care products may include water-based shampoos or dry shampoos. Skin care and hair care products may also include other ingredients, such as UV blockers, moisturizers, humectants, antioxidants, hyaluronic acid, collagen, carriers such as oil and water, and the like.

“Engine” refers to logic embodied in hardware or software instructions, which can be written in a programming language, such as C, C++, COBOL, JAVA™, PHP, Perl, HTML, CSS, JavaScript, VBScript, ASPX, Microsoft .NET™, Go, and/or the like. An engine may be compiled into executable programs or written in interpreted programming languages. Software engines may be callable from other engines or from themselves. Generally, the engines described herein refer to logical modules that can be merged with other engines, or can be divided into sub-engines. The engines can be stored in any type of computer-readable medium or computer storage device and be stored on and executed by one or more general purpose computers, thus creating a special purpose computer configured to provide the engine or the functionality thereof.

“Data store” refers to any suitable device configured to store data for access by a computing device. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network, or may be provided as a cloud-based service. A data store may also include data stored in an organized manner on a computer-readable storage medium, such as a hard disk drive, a flash memory, RAM, ROM, or any other type of computer-readable storage medium. One of ordinary skill in the art will recognize that separate data stores described herein may be combined into a single data store, and/or a single data store described herein may be separated into multiple data stores, without departing from the scope of the present disclosure.

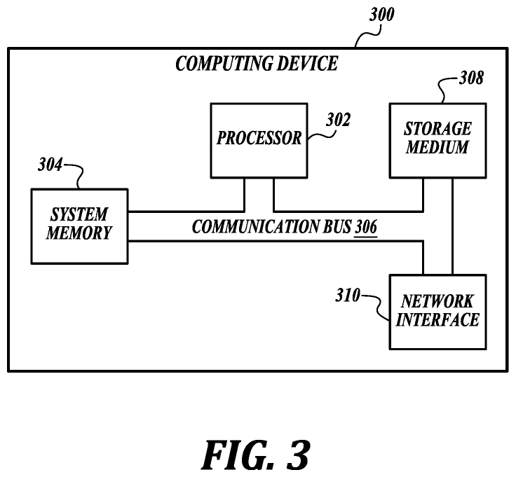

FIG. 3 is a block diagram that illustrates aspects of an exemplary computing device 300 appropriate for use as a mobile computing device of the present disclosure. While multiple different types of computing devices were discussed above, the exemplary computing device 300 describes various elements that are common to many different types of computing devices. While FIG. 3 is described with reference to a mobile computing device, the description below is applicable to servers, personal computers, mobile phones, smart phones, tablet computers, embedded computing devices, and other devices that may be used to implement portions of embodiments of the present disclosure. Moreover, those of ordinary skill in the art and others will recognize that the computing device 300 may be any one of any number of currently available or yet to be developed devices.

In its most basic configuration, the computing device 300 includes at least one processor 302 and a system memory 304 connected by a communication bus 306. Depending on the exact configuration and type of device, the system memory 304 may be volatile or nonvolatile memory, such as read only memory (“ROM”), random access memory (“RAM”), EEPROM, flash memory, or similar memory technology. Those of ordinary skill in the art and others will recognize that system memory 304 typically stores data and/or program modules that are immediately accessible to and/or currently being operated on by the processor 302. In this regard, the processor 302 may serve as a computational center of the computing device 300 by supporting the execution of instructions.

As further illustrated in FIG. 3, the computing device 300 may include a network interface 310 comprising one or more components for communicating with other devices over a network. Embodiments of the present disclosure may access basic services that utilize the network interface 310 to perform communications using common network protocols. The network interface 310 may also include a wireless network interface configured to communicate via one or more wireless communication protocols, such as WiFi, 2G, 3G, LTE, WiMAX, Bluetooth, Bluetooth low energy, and/or the like. As will be appreciated by one of ordinary skill in the art, the network interface 310 illustrated in FIG. 3 may represent one or more wireless interfaces or physical communication interfaces described and illustrated above with respect to particular components of the computing device 300.

In the exemplary embodiment depicted in FIG. 3, the computing device 300 also includes a storage medium 308. However, services may be accessed using a computing device that does not include means for persisting data to a local storage medium. Therefore, the storage medium 308 depicted in FIG. 3 is optional. In any event, the storage medium 308 may be volatile or nonvolatile, removable or nonremovable, implemented using any technology capable of storing information such as, but not limited to, a hard drive, solid state drive, CD ROM, DVD, or other disk storage, magnetic cassettes, magnetic tape, magnetic disk storage, and/or the like.

As used herein, the term “computer-readable medium” includes volatile and non-volatile and removable and non-removable media implemented in any method or technology capable of storing information, such as computer readable instructions, data structures, program modules, or other data. In this regard, the system memory 304 and storage medium 308 depicted in FIG. 3 are merely examples of computer-readable media.

Suitable implementations of computing devices that include a processor 302, system memory 304, communication bus 306, storage medium 308, and network interface 310 are known and commercially available. For ease of illustration and because it is not important for an understanding of the claimed subject matter, FIG. 3 does not show some of the typical components of many computing devices. In this regard, the computing device 300 may include input devices, such as a keyboard, keypad, mouse, microphone, touch input device, touch screen, tablet, and/or the like. Such input devices may be coupled to the computing device 300 by wired or wireless connections including RF, infrared, serial, parallel, Bluetooth, Bluetooth low energy, USB, or other suitable connections protocols using wireless or physical connections. Similarly, the computing device 300 may also include output devices such as a display, speakers, printer, etc. Since these devices are well known in the art, they are not illustrated or described further herein.

FIG. 4 is a flowchart that illustrates a non-limiting example embodiment of a computer-implemented method of generating and providing recommendations of skin care products that supplement vitamin D for a subject. The method 400 may be implemented, in one example, by the mobile computing device 104 alone or in combination with one or more server computing devices 112. The computer-implemented method is performed by the pollutant App 212, pollutant engine 204, occupation engine 206, athletic activity engine 208, recommendation engine 210, user interface engine 214 communicating with each other and with the user data store 202.

In some embodiments, the method may be performed in part by the mobile computing device 104 and in part by the remote server computer system 112. In some embodiments, the mobile computing device 104 is configured to upload data regarding the subject to an external system or server (such as a cloud-based system). Such data may include the user profile. In some embodiments, the method is embodied in the pollutant App 212.

The computer-implemented method 400 may start by clicking on the UV App icon 108 on the display of the mobile computing device 104 to open the UV App 212.

From the start block, the UV App 212 proceeds to block 402, where the UV App 212 receives the subject's 102 profile, location, and occupation or athletic activity, for example.

If a profile has not been provided, the UV App 212 can use the user interface engine 214 to present to the subject, a questionnaire with questions regarding all the relevant information needed to complete the profile. The subject can enter the information through the display device 216. In some embodiments, the UV App 212 accesses the user data store for the profile and other information.

In some embodiments, the location can be constantly determined through GPS in the mobile computing device 104. In this way, the subject's location can be continuously monitored and updated in real time. In some embodiment, the location is used to retrieve information about the pollutant levels at that location.

In some embodiments, the occupation or athletic activity information may be used to determine the wellness program specific to the subject and the relevant pollutant target exposure limits defined in the wellness program. The occupation or athletic activity information can be retrieved from the occupation engine 206 and athletic activity engine 208.

In some embodiments, the pollutant App 212 can be automatically configured to monitor certain pollutants or use different target exposure limits for certain industries or activities. For example, workers in a shipyard can use a differently configured App 212 to workers at a meat processing plant, because workers at a shipyard can be exposed to different pollutants than workers at a meat processing plant. For subjects that work or recreate outdoors, the App 21 can be configured differently to other workers that may work indoors. For example, temperature and humidity can be monitored for subjects that work outdoors . From block 402, the method proceeds to block 404.

In block 404, the pollutant engine 204 retrieves the amount of pollutants at the subject's location. The amount of pollutants can be retrieved from online sources on air quality or the amount of pollutants can be determined by one or more sensors 106 worn by the subject 102 or placed on the mobile computing device 104. Depending on the sensor 106, data can be processed by the sensor 106 or the mobile computing device 104. In some embodiments, the subject 102 scans the sensor 106 with the mobile computing device 104 to establish a connection between the sensor 106 and the mobile computing device 104. Communication pairing is performed between the sensor 106 and the mobile computing device 104 when the two devices are within an acceptable wireless communication range of each other. In some embodiments, the sensor 106 includes RFID and antenna for the subject to obtain the data wirelessly.

To illustrate how a pollutant sensor may operate, an example is provided where the sensor 106 is a UV sensor that contains a UV sensitive LED that will induce electronic current proportional to UV exposure. The amount of UV exposure can then be converted and stored as voltage, which is a measurement of cumulative UV exposure over time. UV exposure can be reported on a per unit of time basis, such as daily, weekly, monthly, etc. The voltage is read each time as the subject scans the sensor 106. The scanned voltage data is converted into a UV-A dosage based on the calibrated correlations. In some embodiments, UV-B exposure is then calculated using a pre-computed lookup table that gives the conversion factor as function of the column amount of ozone in the atmosphere and solar zenith angle (SZA). SZA is determined based on GPS location and time. Other sensors capable of measuring the amount of other pollutants can be similarly configured to be read by the mobile computing device 104. From block 404, the method proceeds to block 406.

In block 406, whether the pollutant engine 204 receives the pollutant levels from sensors 106 or online sources, the pollutant engine 204 keeps track of the pollutant levels at the subject's location and every time the location changes, and the time at the locations to integrate the pollutant levels into a running exposure amount over time. In this manner, the pollutant engine 204 can keep track of the subject's location and the pollutant levels at each location throughout the subject's daily routine. This can be done automatically by the mobile computing device 104, or the subject can decide when to turn the pollutant App 212 on and off. The subject 102 can also follow their pollutant level exposure over time. The pollutant engine 204 can keep a running total of pollutant exposure in any increments of time, such as by the minute, hour, day, week, month, or year. From block 406, the method proceeds to block 408.

In block 408, the pollutant engine 204 acquires the target exposure limits from the occupation engine 206 or the athletic activity engine 208 and other information to compare to the integrated pollutant exposure amounts. As mentioned, target exposure limits can be adjusted for each subject, or more than one target exposure limit can be used to compare against the integrated pollution exposure level for different purposes, such as to provide product recommendations or warning notices. From block 408, the method enters block 410.

In block 410, the pollutant engine 204 logs the trends of the pollutant exposure amount compared to the pollutant target limit for each pollutant. In some embodiments, the pollutant exposure amount can be tracked as a percentage compared to the pollutant target limit. In this manner, the pollutant engine 204 can determine those pollutants to which the subject has the highest exposure. From block 410, the method proceeds to block 412.

In block 412, the recommendation engine 210 makes a personalized set of skin and hair care product recommendations based on the subject's highest trending pollutants. The recommendations can consider any number of the highest trending pollutants, for example, the recommendation engine 210 can base its product recommendations based on the three to the highest trending pollutants. However, the number of pollutants can be greater, such as twenty or more. In some embodiments, the product recommendations is based on those pollutants that have reached a percentage of the target exposure limit. For example, product recommendations can be based on those pollutants to which the subject has reached 50%, 75%, 90%, etc. of the target exposure limit.

In block 412, the user interface engine 214 may create tutorials on how to use the skin care products. The user interface engine 214 may create and download protocols for a regimen or routine. The user interface engine 214 may can coach, track usage and compare the tracked usage to the protocol, the regimen, and the routine. Additionally, the user interface engine 214 can be used to make a purchase of any products related to skincare or hair care. From block 412, the method proceeds to block 414.

In block 414, the pollutant engine 204 can use the same or different target exposure limits. In block 414, the pollutant engine 204 makes a determination for each pollutant whether or not the integrated pollutant amount to which the subject has been exposed is greater than the target exposure limit on a per time basis. For example, the pollutant engine 204 compares the current integrated exposure amount of a pollutant to determine whether that pollutant exceeds the target exposure limit per minute, per hour, per day, per week, per month, per year or per lifetime as determined by the occupation engine 206 or the athletic activity engine 208. In block 414, the target exposure limit of a pollutant may coincide with the permissible exposure limit of that pollutant as determined by a government entity, which are determined primarily for health reasons. The target exposure limit for block 414 can also come from the occupation or athletic activity engines 206, 208. Therefore, the purpose of block 414 can be for a health and wellness purpose of the subject as compared to the product recommendation of block 412. If the pollutant engine 204 determines that an integrated exposure amount of a pollutant has exceeded the target exposure limit for that pollutant, then, the method enters block 416.

In block 416, the recommendation engine 210 can display a notification to the subject the a target exposure limit has been exceeded and may also recommend a corrective action. Foe example, corrective actions related to an occupation or athletic activity can include leaving the area, donning personal protective equipment, changing or reducing work schedules, changing or reducing athletic practice schedules, and provide other useful help and information.

The user interface engine 214 can determine the appropriate user interface displayed by the mobile computing device 104. In addition, the user interface engine 214 can also provide graphs, data, information, warnings, useful links, and help. In some embodiments, the user interface engine 214 may create a display on the mobile computing device 104 with an indication of the user risk in percentage form, along with a category label such as “low”, “moderate,” or “high.” A graph may also be displayed that tracks the pollutant exposure levels over time. The subject may recall any prior history on exposure levels for the pollutants.

If the pollutant engine 204 determines that an integrated exposure amount of a pollutant has not exceeded the target exposure limit, then, the method returns to block 404 to continue to acquire and integrate the pollutant amounts for the subject, and continues to make product recommendation as in block 412 or notifications of the risk of over exposure to pollutants in accordance with occupational or athletic wellness programs.

Representative embodiments are given by the following examples.

In one embodiment, a computer-implemented method of generating and providing skin or hair care product recommendations to a subject comprises determining, by a computing device, more than one pollutant exposure amount for the subject; determining, by the computing device, a target exposure limit for each pollutant; determining, by the computing device, pollutants to which the subject has the highest exposure; and providing, by the computing device, at least one skin or hair care product recommendation to the subject, wherein the recommendation is based on the pollutants to which the subject has the highest exposure.

In one embodiment, the computer-implemented method further comprises providing a notification that the subject is at risk from exposure to a pollutant over a target exposure limit.

In one embodiment, the pollutants to which the subject has the highest exposure are determined based on comparing the percentages of exposure amount of each pollutant to a target exposure limit for each pollutant.

In one embodiment, the computer-implemented method further comprises integrating a pollutant exposure amount as a subject changes location.

In one embodiment, the computer-implemented method further comprises receiving pollutant amount data from a wearable sensor or an online source or both.

In one embodiment, the target exposure limit is selected based on recommending a skin or hair care product or based on recommending a corrective action based on health or wellness.

In one embodiment, the computer-implemented method further comprises receiving a target exposure limit based on a subject's profile.

In one embodiment, the target exposure limit is based on a subject's occupation or athletic activity.

In one embodiment, a computing device is configured to determine more than one pollutant exposure amount for a subject; determine a target exposure limit for each pollutant; determine pollutants to which the subject has the highest exposure; and provide at least one skin or hair care product recommendation to the subject, wherein the recommendation is based on the pollutants to which the subject has the highest exposure.

In one embodiment, the computing device is further configured to provide a notification that the subject is at risk from exposure to a pollutant over a target exposure limit.

In one embodiment, the pollutants to which the subject has the highest exposure are determined based on comparing the percentages of exposure amount of each pollutant to a target exposure limit for each pollutant.

In one embodiment, the computing device is further configured to integrate a pollutant exposure amount as a subject changes location.

In one embodiment, the computing device is further configured to receive pollutant amount data from a wearable sensor or an online source or both.

In one embodiment, the target exposure limit is selected based on recommending a skin or hair care product or based on recommending a corrective action based on health or wellness.

In one embodiment, the computing device is further configured to receive a target exposure limit based on a subject's profile.

In one embodiment, the target exposure limit is based on a subject's occupation or athletic activity.

In one embodiment, a system comprises an occupational or athletic activity engine including computational circuitry configured to: determine a target exposure limit for each pollutant; a pollutant engine including computational circuitry configured to: determine more than one pollutant exposure amount for a subject; determine pollutants to which the subject has the highest exposure; and a recommendation engine including computational circuitry configured to: provide at least one skin or hair care product recommendation to the subject, wherein the recommendation is based on the pollutants to which the subject has the highest exposure.

In one embodiment, the system further comprises a user interface engine including computational circuitry configured to: provide a notification that the subject is at risk from exposure to a pollutant over a target exposure limit.

In one embodiment, the pollutants to which the subject has the highest exposure are determined based on comparing the percentages of exposure amount of each pollutant to a target exposure limit for each pollutant.

In one embodiment, the pollutant engine further comprises computational circuitry configured to: integrate a pollutant exposure amount as a subject changes location.

In one embodiment, the system is further configured to receive pollutant amount data from a wearable sensor or an online source or both.

In one embodiment, the target exposure limit is selected based on recommending a skin or hair care product or based on recommending a corrective action based on health or wellness.

In one embodiment, the system is further configured to receive a target exposure limit based on a subject's profile.

In one embodiment, the system further comprises an occupational engine including computational circuitry configured to: provide a target exposure limit based on a subject's occupation; or an athletic activity engine including computation circuitry configured to: provide a target exposure limit based on a subject's athletic activity.

While illustrative embodiments have been illustrated and described, it will be appreciated that various changes can be made therein without departing from the spirit and scope of the invention.

The embodiments of the invention in which an exclusive property or privilege is claimed are defined as follows:

1. A computer-implemented method of generating and providing skin or hair care product recommendations to a subject, the method comprising:
determining, by a computing device, more than one pollutant exposure amount for the subject;
determining, by the computing device, a target exposure limit for each pollutant;
determining, by the computing device, pollutants to which the subject has the highest exposure; and
providing, by the computing device, at least one skin or hair care product recommendation to the subject, wherein the recommendation is based on the pollutants to which the subject has the highest exposure.

2. The computer-implemented method of claim 1, further comprising providing a notification that the subject is at risk from exposure to a pollutant over a target exposure limit.

3. The computer-implemented method of claim 1, wherein the pollutants to which the subject has the highest exposure are determined based on comparing the percentages of exposure amount of each pollutant to a target exposure limit for each pollutant.

4. The computer-implemented method of claim 1, further comprising integrating a pollutant exposure amount as a subject changes location.

5. The computer-implemented method of claim 1, further comprising receiving pollutant amount data from a wearable sensor or an online source or both.

6. The computer-implemented method of claim 1, wherein the target exposure limit is selected based on recommending a skin or hair care product or based on recommending a corrective action based on health or wellness.

7. The computer-implemented method of claim 1, further comprising receiving a target exposure limit based on a subject's profile.

8. The computer-implemented method of claim 1, wherein the target exposure limit is based on a subject's occupation or athletic activity.

9. A computing device configured to:
determine more than one pollutant exposure amount for a subject;
determine a target exposure limit for each pollutant;
determine pollutants to which the subject has the highest exposure; and
provide at least one skin or hair care product recommendation to the subject, wherein the recommendation is based on the pollutants to which the subject has the highest exposure.

10. The computing device of claim 9, wherein the computing device is further configured to provide a notification that the subject is at risk from exposure to a pollutant over a target exposure limit.

11. The computing device of claim 9, wherein the pollutants to which the subject has the highest exposure are determined based on comparing the percentages of exposure amount of each pollutant to a target exposure limit for each pollutant.

12. The computing device of claim 9, wherein the computing device is further configured to integrate a pollutant exposure amount as a subject changes location.

13. The computing device of claim 9, wherein the computing device is further configured to receive pollutant amount data from a wearable sensor or an online source or both.

14. The computing device of claim 9, wherein the target exposure limit is selected based on recommending a skin or hair care product or based on recommending a corrective action based on health or wellness.

15. The computing device of claim 9, wherein the computing device is further configured to receive a target exposure limit based on a subject's profile.

16. The computing device of claim 9, wherein the target exposure limit is based on a subject's occupation or athletic activity.

17. A system, comprising:
an occupational or athletic activity engine including computational circuitry configured to:
determine a target exposure limit for each pollutant;
a pollutant engine including computational circuitry configured to:
determine more than one pollutant exposure amount for a subject;
determine pollutants to which the subject has the highest exposure; and
a recommendation engine including computational circuitry configured to:
provide at least one skin or hair care product recommendation to the subject, wherein the recommendation is based on the pollutants to which the subject has the highest exposure.

18. The system of claim 17, wherein the system further comprises a user interface engine including computational circuitry configured to:
provide a notification that the subject is at risk from exposure to a pollutant over a target exposure limit.

19. The system of claim 17, wherein the pollutants to which the subject has the highest exposure are determined based on comparing the percentages of exposure amount of each pollutant to a target exposure limit for each pollutant.

20. The computing device of claim 17, wherein the pollutant engine further comprises computational circuitry configured to:
integrate a pollutant exposure amount as a subject changes location.

21. The computing device of claim 17, wherein the system is further configured to receive pollutant amount data from a wearable sensor or an online source or both.

22. The computing device of claim 17, wherein the target exposure limit is selected based on recommending a skin or hair care product or based on recommending a corrective action based on health or wellness.

23. The computing device of claim 17, wherein the system is further configured to receive a target exposure limit based on a subject's profile.

24. The computing device of claim 17, wherein the system further comprises;
an occupational engine including computational circuitry configured to:
provide a target exposure limit based on a subject's occupation; or
an athletic activity engine including computation circuitry configured to:
provide a target exposure limit based on a subject's athletic activity.
